(19) United States
(12) Patent Application Publication
(10) Pub. No.: US 2015/0190319 A1
(43) Pub. Date: Jul. 9, 2015

(54) COSMETIC COMPOSITION OF HYDROPHOBIC SILICA AEROGEL PARTICLES AND A POLYMER COMPRISING A SUGAR UNIT

(71) Applicant: L'OREAL, Paris (FR)

(72) Inventors: Samira Khenniche, Clichy (FR); Gregory Plos, Paris (FR)

(73) Assignee: L'OREAL, Paris (FR)

(21) Appl. No.: 14/409,913

(22) PCT Filed: Jun. 20, 2013
(86) PCT No.: PCT/FR2013/051437

§ 371 Date: Dec. 19, 2014

Related U.S. Application Data
(60) Provisional application No. 61/727,144, filed on Nov. 16, 2012.

(30) Foreign Application Priority Data
Jun. 21, 2012 (FR) 1255815

(51) Int. Cl. A61K 8/25; A61Q 5/02; A61Q 5/00; A61Q 5/12; A61K 8/97; A61K 8/73

(52) U.S. Cl. CPC A61K 8/25; A61K 8/97; A61K 8/731; A61Q 5/008; A61Q 5/12; A61Q 5/02; A61K 2800/412; A61K 2800/28

(57) ABSTRACT

The present invention relates to a cosmetic composition comprising hydrophobic silica aerogel particles and at least one polymer bearing a sugar unit.

The invention also relates to a cosmetic treatment method using said composition, especially a hair treatment method that makes it possible in particular to delay or even prevent regreasing of the hair and/or of the scalp.

The present invention relates to a cosmetic composition, in particular a hair composition, comprising the combination of hydrophobic silica aerogel particles and particular thickening polymers, and also to a method of using said composition.

Hair has a tendency to lose some of its qualities due to the action of factors such as natural regreasing, sweat, the removal of squamae, pollution, humidity and other factors. The visual appearance or the feel of the hair can thus be damaged. Regreasing, for example due to pollution, makes the hair lank, which then has a tendency to clump together. The hair may be difficult to style, in addition to having, for example, an unpleasant waxy feel or greasy sheen. In order to combat these inconveniences, it is possible to use detergent compositions, for example shampoos, in order to remove contaminants (sebum, sweat, pollution, etc.) or dandruff, and to loosen the hair. The hair is subsequently rinsed and then dried. The shampooing operations have to be repeated regularly, for example after a few days, or even after a few hours. However, shampoos, based on large amounts of surfactants, may cause discomfort such as stinging on the scalp or in the eyes. Shampoo compositions or compositions for washing the skin may also combine, with the surfactants, sebum absorbers in order to make it possible to extend in time the perception of cleanliness of the hair or skin. However, it has been found that the effectiveness of these products is still insufficient with respect to the expectations of consumers. This is because they do not make it possible to significantly reduce the frequency of washing of the hair in particular, which frequency may differ depending on the country or even the region in question and which may range from one to two shampooing operations per day to one or two shampooing operations per week.

To date, no hygienic cosmetic composition, in particular hair composition, makes it possible to significantly slow down regreasing, in particular of the hair.

The objective of the present invention is to provide cosmetic compositions that overcome these disadvantages.

The compositions according to the invention make it possible to retain a perception of clean hair for a longer time than with a standard shampoo; by way of example, this perception of clean hair may be one week for people who normally wash their hair 2 to 3 times a week, and may be at least 3 days for people who normally wash their hair every day.

Furthermore, the compositions according to the invention make it possible to obtain cleansing performances at least identical to those of a standard shampoo, in particular a very good detergency power.

One subject of the invention is therefore a cosmetic composition comprising hydrophobic silica aerogel particles and at least one polymer bearing a sugar unit or units.

It is been found that the use of the compositions according to the invention makes it possible to significantly reduce regreasing of the hair and/or of the scalp, and thus makes it possible to reduce the frequency of washing.

In addition, the composition according to the invention makes it possible to obtain good hair conditioning properties, especially in terms of softness, manageability, smoothing and disentangling, while having improved distribution and spreading over the hair.

In the present description, the expression “at least one” is equivalent to the expression “one or more” and can be replaced therewith.

In the present description, the expression “between” is equivalent to the expression “ranging from” and can be replaced therewith.

Hydrophobic Silica Aerogel Particles

The composition according to the invention therefore comprises hydrophobic silica aerogel particles.

Aerogels are ultra-light porous materials, the first ones of which were made by Kristler in 1932.

They are generally synthesized via a sol-gel process in liquid medium and then dried by extraction of a supercritical fluid. The supercritical fluid most commonly used is supercritical CO₂. This type of drying makes it possible to avoid shrinkage of the pores and of the material. Other types of drying also make it possible to obtain porous materials from gel, namely for example (i) drying by cryodesiccation, which consists in solidifying the gel at low temperature and then in subliming off the solvent, and (ii) drying by evaporation. The materials thus obtained are then known, respectively, as cryogels and xerogels. The sol-gel process and the various drying operations are described in detail in Brinker C J., and Scherer G. W., Sol-Gel Science, New York, Academic Press, 1990.

The term “hydrophobic silica” means any silica whose surface is treated with silylating agents, for example halogenated silanes such as alkylchlorosilanes, siloxanes, in particular dimethylsiloxanes such as hexamethyldisiloxane, or silazanes, so as to functionalize the OH groups with silyl groups Si—Rn, for example trimethylsilyl groups.

Preferably, the hydrophobic silica aerogel particles capable of being used in the present invention advantageously have a specific surface area per unit of mass (S_{M}) ranging from 500 to 1500 m²/g, preferably from 600 to 1200 m²/g and better still from 600 to 800 m²/g.

Preferably, the hydrophobic silica aerogel particles capable of being used in the present invention advantageously have an oil absorption capacity, measured at the wet point, ranging from 5 to 18 ml/g, preferably from 6 to 15 ml/g and better still from 8 to 12 ml/g of particles.

Preferably, the hydrophobic silica aerogel particles capable of being used in the present invention advantageously have a size, expressed as the mean diameter (D[0.5]), of less than 1500 μm, and preferably ranging from 1 to 30 μm, preferably from 5 to 25 μm, better still from 5 to 20 μm and even better still from 5 to 15 μm.

Preferably, the hydrophobic silica aerogel particles capable of being used in the present invention advantageously have a tapped density p ranging from 0.04 g/cm³ to 0.10 g/cm³ and preferably from 0.05 g/cm³ to 0.08 g/cm³.

Preferably, the hydrophobic silica aerogel particles capable of being used in the present invention advantageously have a specific surface area per unit of volume (S_{V}) ranging from 5 to 60 m²/cm³, preferably from 10 to 50 m²/cm³ and better still from 15 to 40 m²/cm³.

According to one preferred embodiment, the hydrophobic silica aerogel particles according to the invention have a specific surface area per unit of mass (S_{M}) ranging from 500 to 1500 m²/g, preferably from 600 to 1200 m²/g and better still from 600 to 800 m²/g, and have a size, expressed as the mean diameter (D[0.5]), ranging from 1 to 30 μm and/or an oil absorption capacity, measured at the wet point, ranging from 5 to 18 ml/g, preferably from 6 to 15 ml/g and better still from 8 to 12 ml/g of particles.

According to another preferred embodiment, the hydrophobic silica aerogel particles used in the present invention have a specific surface area per unit of mass (S_{M}) ranging from 600 to 800 m²/g and a size, expressed as the volume mean diameter (D[0.5]), ranging from 5 to 20 μm and better still from 5 to 15 μm.

The specific surface area per unit of mass can be determined by the nitrogen absorption method, known as the BET (Brunauer-Emmett-Teller) method, described in The Journal of the American Chemical Society, vol. 60, page 309, February 1938, which corresponds to international standard ISO 5794/1 (appendix D). The BET specific surface area corresponds to the total specific surface area of the particles under consideration.

The absorption capacity measured at the wet point, denoted Wp, corresponds to the amount of oil which it is necessary to add to 100 g of particles in order to obtain a homogeneous paste. It is measured according to the “wet point” method or the method for determining the oil uptake of a powder according to the principle described in standard NF T 30-022. It corresponds to the amount of oil adsorbed onto the available surface of the powder and/or absorbed by the powder by measurement of the wet point, described below:

An amount m=2 g of powder is placed on a glass plate, and the oil (isononyl isononanoate) is then added dropwise. After addition of 4 to 5 drops of oil to the powder, mixing is carried out using a spatula, and addition of oil is continued until conglomerates of oil and powder have formed. From this point, the oil is added at the rate of one drop at a time and the mixture is subsequently triturated with the spatula. The addition of oil is stopped when a firm, smooth paste is obtained. This paste must be able to be spread on the glass plate without cracking or forming lumps. The volume Vs (expressed in ml) of oil used is then noted.

The oil uptake (oil absorption capacity) corresponds to the ratio Vs/m.

The sizes of the aerogel particles according to the invention may be measured by static light scattering using a commercial particle size analyzer such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is especially described in the publication by Van de Hulst, H. C., “Light Scattering by Small Particles”, Chapters 9 and 10, Wiley, New York, 1957.

In the context of the present invention, the tapped density may be assessed according to the following protocol, known as the tapped density protocol: 40 g of powder are poured into a measuring cylinder and the cylinder is then placed on a Stay 2003 machine from Stampf Volumeter. The cylinder is then subjected to a series of 2500 tapping actions (this operation is repeated until the difference in volume between two consecutive tests is less than 2%); the final volume Vf of tapped powder is then measured directly on the cylinder. The tapped density is determined by the ratio: mass (m)/Vf, in this instance 40/Vf (Vf being expressed in cm³ and m in g).

The specific surface area per unit of volume is given by the relationship: S_{V}=S_{M}×ρ, where ρ is the tapped density, expressed in g/cm³, and S_{M} is the specific surface area per unit of mass, expressed in m²/g, as defined above.

The hydrophobic silica aerogel particles used according to the present invention are preferably silylated silica (INCI name: silica silylate) aerogel particles. The preparation of hydrophobic silica aerogel particles that have been surface-modified by silylation is described more fully in U.S. Pat. No. 7,470,725. Use will be made in particular of aerogel particles of hydrophobic silica surface-modified with trimethylsilyl groups.

As hydrophobic silica aerogels that may be used in the invention, examples that may be mentioned include the aerogel sold under the name VM-2260 (INCI name: silica silylate), by the company Dow Corning, the particles of which have an average size of about 1000 microns and a specific surface area per unit of mass ranging from 600 to 800 m²/g.

Mention may also be made of the aerogels sold by the company Cabot under the references Aerogel TLD 201, Aerogel OGD 201 and Aerogel TLD 203, Enova Aerogel MT 1100 and Enova Aerogel MT 1200.

Use will be made more particularly of the aerogel sold under the name VM-2270 (INCI name: silica silylate), by the company Dow Corning, the particles of which have an average size ranging from 5 to 15 microns and a specific surface area per unit of mass ranging from 600 to 800 m²/g.

A mixture of hydrophobic silica aerogel particles may, of course, be used.

The compositions according to the invention may comprise the hydrophobic silica aerogel particles in an amount of between 0.01% and 20% by weight, preferably between 0.05% and 10% by weight and preferentially between 0.1% and 5% by weight, relative to the total weight of the composition.

Polymers Bearing a Sugar Unit

The composition according to the invention also comprises at least one polymer bearing sugar unit(s). A mixture of such polymers may obviously be used.

Preferably, the polymer bearing sugar unit(s) is a thickening polymer. For the purposes of the present invention, the term “thickening polymer” means a polymer which, when introduced at 1% by weight in an aqueous solution or an aqueous-alcoholic solution containing 30% ethanol, and at pH 7, makes it possible to achieve a viscosity of at least 100 cps and preferably of at least 500 cps, at 25° C. and at a shear rate of 1 s⁻¹. This viscosity may be measured using a cone/plate viscometer (Haake R600 rheometer or the like).

The term “sugar unit” means a unit derived from a carbohydrate of formula Cₙ(H₂O)ₙ₋₁ or (CH₂O)ₙ, which may be optionally modified by substitution and/or by oxidation and/or by dehydration.

The sugar units that may be included in the composition of the polymers of the invention are preferably derived from the following sugars: glucose, galactose, arabinose, rhamnose, mannose, xylose, fucose, fructose, anhydrogalactose, galacturonic acid, glucuronic acid, mannuronic acid, galactose sulfate, anhydrogalactose sulfate.

The polymers bearing sugar unit(s) according to the invention may be of natural or synthetic origin. They may be nonionic, anionic, amphoteric or cationic. The base units of the polymers bearing a sugar unit of the invention may be monosaccharides or disaccharides.

As polymers that may be used, mention may be made especially of the following native gums, and also derivatives thereof:

a) tree or shrub exudates, including:
gum arabic (branched polymer of galactose, arabinose, rhamnose and glucuronic acid);
ghatti gum (polymer derived from arabinose, galactose, mannose, xylose and glucuronic acid);
karaya gum (polymer derived from galacturonic acid, galactose, rhamnose and glucuronic acid);
gum tragacanth (or tragacanth) (polymer of galacturonic acid, galactose, fucose, xylose and arabinose);

b) gums derived from algae, including:
agar (polymer derived from galactose and anhydrogalactose);
alginates (polymers of mannuronic acid and of glucuronic acid);
carrageenans and furcellerans (polymers of galactose sulfate and of anhydrogalactose sulfate);

c) gums derived from seeds or tubers, including:
guar gum (polymer of mannose and galactose);
locust bean gum (polymer of mannose and galactose);
fenugreek gum (polymer of mannose and galactose);
tamarind gum (polymer of galactose, xylose and glucose);
konjac gum (polymer of glucose and mannose);

d) microbial gums, including:
xanthan gum (polymer of glucose, mannose acetate, mannose/pyruvic acid and glucuronic acid);
gellan gum (polymer of partially acylated glucose, rhamnose and glucuronic acid);
scleroglucan gum (glucose polymer);

e) plant extracts, including:
cellulose (glucose polymer);
starch (glucose polymer);
inulin (polymer of fructose and glucose).

These polymers may be physically or chemically modified. A physical treatment that may especially be mentioned is the temperature. Chemical treatments that may be mentioned include esterification, etherification, amidation or oxidation reactions. These treatments can lead to polymers that may be nonionic, anionic, cationic or amphoteric.

Preferably, these chemical or physical treatments are applied to guar gums, locust bean gums, starches and celluloses.

The nonionic guar gums that may be used according to the invention may be modified with C1-C6 hydroxyalkyl groups. Among the hydroxyalkyl groups that may be mentioned are hydroxymethyl, hydroxyethyl, hydroxypropyl and hydroxybutyl groups.

These guar gums are well known in the prior art and may be prepared, for example, by reacting the corresponding alkene oxides, for instance propylene oxides, with the guar gum so as to obtain a guar gum modified with hydroxypropyl groups.

The degree of hydroxyalkylation preferably ranges from 0.4 to 1.2, and corresponds to the number of alkylene oxide molecules consumed by the number of free hydroxyl functions present on the guar gum.

Such nonionic guar gums optionally modified with hydroxyalkyl groups are sold, for example, under the trade names Jaguar HP8, Jaguar HP60 and Jaguar HP120 by the company Rhodia Chimie.

The guar gums modified with cationic groups that may be used more particularly according to the invention are guar gums comprising trialkylammonium cationic groups. Preferably, 2% to 30% by number of the hydroxyl functions of these guar gums bear trialkylammonium cationic groups. Even more preferentially, 5% to 20% by number of the hydroxyl functions of these guar gums are branched with trialkylammonium cationic groups. Among these trialkylammonium groups, mention may be made most particularly of trimethylammonium and triethylammonium groups. Even more preferentially, these groups represent from 5% to 20% by weight relative to the total weight of the modified guar gum. According to the invention, use may be made of guar gums modified with 2,3-epoxypropyltrimethylammonium chloride.

These guar gums modified with cationic groups are products already known per se and are, for example, described in patents U.S. Pat. No. 3,589,578 and U.S. Pat. No. 40,131,307. Such products are moreover sold especially under the trade names Jaguar C13 S, Jaguar C 15 and Jaguar C 17 by the company Rhodia Chimie.

A modified locust bean gum that may be used is cationic locust bean gum containing hydroxypropyltrimonium groups, such as Catinal CLB 200 sold by the company Toho.

The starch molecules used in the present invention may originate from any plant source of starch, especially cereals and tubers; more particularly, they may be starches from corn, rice, cassava, barley, potato, wheat, sorghum, pea, oat or tapioca. It is also possible to use the hydrolysates of the starches mentioned above. The starch is preferably derived from potato.

The starches may be chemically or physically modified, especially by one or more of the following reactions: pregelatinization, oxidation, crosslinking, esterification, etherification, amidation, heat treatments.

More particularly, these reactions may be performed in the following manner:
pregelatinization by splitting the starch granules (for example drying and cooking in a drying drum);
oxidation with strong oxidizing agents, leading to the introduction of carboxyl groups into the starch molecule and to depolymerization of the starch molecule (for example by treating an aqueous starch solution with sodium hypochlorite);
crosslinking with functional agents capable of reacting with the hydroxyl groups of the starch molecules, which will thus be bonded together (for example with glyceryl and/or phosphate groups);
esterification in alkaline medium for the grafting of functional groups, especially C1-C6 acyl(acetyl), C1-C6 hydroxyalkyl(hydroxyethyl or hydroxypropyl), carboxymethyl or octenylsuccinic.

Monostarch phosphates (of the type Am—O—PO—(OX)₂), distarch phosphates (of the type Am—O—PO—(OX)—O—Am) or even tristarch phosphates (of the type Am—O—PO—(O—Am)₂) or mixtures thereof may especially be obtained by crosslinking with phosphorus compounds, Am meaning starch and X especially denoting alkali metals (for example sodium or potassium), alkaline earth metals (for example calcium or magnesium), ammonium salts, amine salts, for instance those of monoethanolamine, diethanolamine, triethanolamine, 3-amino-1,2-propanediol, or ammonium salts derived from basic amino acids such as lysine, arginine, sarcosine, ornithine or citrulline.

The phosphorus compounds may be, for example, sodium tripolyphosphate, sodium orthophosphate, phosphorus oxychloride or sodium trimetaphosphate. Distarch phosphates or compounds rich in distarch phosphate will preferentially be used, for instance the product sold under the references Prejel VA-70-T AGGL (gelatinized hydroxypropyl cassava distarch phosphate), Prejel TK1 (gelatinized cassava distarch phosphate) and Prejel 200 (gelatinized acetyl cassava distarch phosphate) by the company Avebe, or Structure Zea from National Starch (gelatinized corn distarch phosphate).

A preferred starch is a starch that has undergone at least one chemical modification such as at least one esterification.

According to the invention, amphoteric starches comprising one or more anionic groups and one or more cationic groups may also be used. The anionic and cationic groups may be linked to the same reactive site of the starch molecule or to different reactive sites; they are preferably linked to the same reactive site. The anionic groups may be of carboxylic, phosphate or sulfate type, preferably carboxylic. The cationic groups may be of primary, secondary, tertiary or quaternary amine type.

The amphoteric starches are especially chosen from the compounds having the following formulae:

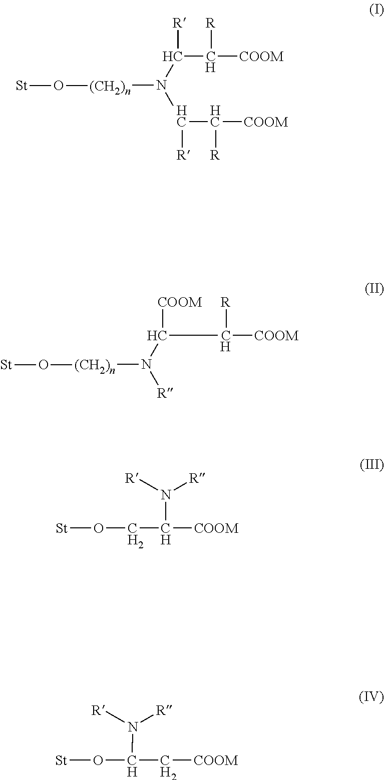

in which:

St-O represents a starch molecule,

R, which may be identical or different, represents a hydrogen atom or a methyl radical,

R′, which may be identical or different, represents a hydrogen atom, a methyl radical or a —COON group,

n is an integer equal to 2 or 3,

M, which may be identical or different, denotes a hydrogen atom, an alkali metal or alkaline earth metal such as Na, K, Li or NH4, a quaternary ammonium or an organic amine,

R″ represents a hydrogen atom or a C1-C18 alkyl radical.

These compounds are especially described in U.S. Pat. No. 5,455,340 and U.S. Pat. No. 4,017,460.

Use is particularly made of the starches of formulae (II) or (Ill); and preferentially starches modified with 2-chloroethylaminodipropionic acid, i.e. starches of formula (II) or (III) in which R, R′, R″ and M represent a hydrogen atom and n is equal to 2. The preferred amphoteric starch is a starch chloroethylamidodipropionate.

The celluloses and cellulose derivatives may be anionic, cationic, amphoteric or nonionic. Among these derivatives, cellulose ethers, cellulose esters and cellulose ester ethers are distinguished.

Among the cellulose esters, mention may be made of inorganic cellulose esters (cellulose nitrates, sulfates and phosphates), organic cellulose esters (cellulose monoacetates, triacetates, amidopropionates, acetate butyrates, acetate propionates and acetate trimellitates), and mixed organic/inorganic cellulose esters, such as cellulose acetate butyrate sulfates and cellulose acetate propionate sulfates.

Among the cellulose ester ethers, mention may be made of hydroxypropyl methyl cellulose phthalates and ethyl cellulose sulfates.

Among the nonionic cellulose ethers that may be mentioned are alkyl celluloses such as methyl celluloses and ethyl celluloses (for example Ethocel Standard 100 Premium from Dow Chemical); hydroxyalkyl celluloses such as hydroxymethyl celluloses and hydroxyethyl celluloses (for example Natrosol 250 HHR sold by Aqualon) and hydroxypropyl celluloses (for example Klucel EF from Aqualon); mixed hydroxyalkyl alkyl celluloses such as hydroxypropyl methyl celluloses (for example Methocel E4M from Dow Chemical), hydroxyethyl methyl celluloses, hydroxyethyl ethyl celluloses (for example Bermocoll E 481 FQ from Akzo Nobel) and hydroxybutyl methyl celluloses.

Among the anionic cellulose ethers, mention may be made of carboxyalkyl celluloses and salts thereof. Examples that may be mentioned include carboxymethyl celluloses, carboxymethyl methyl celluloses (for example Blanose 7M from the company Aqualon) and carboxymethyl hydroxyethyl celluloses, and also the sodium salts thereof.

Among the cationic cellulose ethers, mention may be made of crosslinked or non-crosslinked, quaternized hydroxyethyl celluloses. The quaternizing agent may especially be diallyldimethylammonium chloride (for example Celquat L200 from National Starch). Another cationic cellulose ether that may be mentioned is hydroxypropyltrimethylammonium hydroxyethyl cellulose (for example Ucare Polymer JR 400 from Amerchol).

Among the associative thickening polymers bearing sugar unit(s), mention may be made of celluloses or derivatives thereof, modified with groups comprising at least one fatty chain, such as alkyl, arylalkyl or alkylaryl groups or mixtures thereof in which the alkyl groups are C8-C22 alkyl groups; nonionic alkylhydroxyethyl celluloses such as the products Natrosol Plus Grade 330 CS and Polysurf 67 (C16 alkyl) sold by the company Aqualon; (cationic) quaternized alkylhydroxyethyl celluloses, such as the products Quatrisoft LM 200, Quatrisoft LM-X 529-18-A, Quatrisoft LM-X 529-18-B (C12 alkyl) and Quatrisoft LM-X 529-8 (C18 alkyl) sold by the company Amerchol, the products Crodacel QM and Crodacel QL (C12 alkyl) and Crodacel QS (C18 alkyl) sold by the company Croda, and the product Softcat SL 100 sold by the company Amerchol; nonionic nonoxynylhydroxyethyl celluloses such as the product AmerceII HM-1500 sold by the company Amerchol; nonionic alkyl celluloses such as the product Bermocoll EHM 100 sold by the company Berol Nobel.

As associative polymers bearing sugar unit(s) derived from guar, mention may be made of hydroxypropyl guars modified with a fatty chain, such as the product Esaflor HM 22 (modified with a C22 alkyl chain) sold by the company Lamberti; the product Miracare XC 95-3 (modified with a C14 alkyl chain) and the product RE 205-146 (modified with a C20 alkyl chain) sold by Rhodia Chimie.

The polymer(s) bearing sugar unit(s) of the invention are preferably chosen from guar gums, locust bean gums, xanthan gums, starches and celluloses, in their modified form (derivatives) or unmodified form.

Preferably, the polymers bearing sugar unit(s) according to the invention are nonionic.

The composition according to the invention comprises the polymer(s) bearing sugar unit(s) preferably in an amount of between 0.01% and 10% by weight, especially from 0.05% to 5% by weight, preferentially from 0.1% to 1% by weight, or even from 0.1% to 0.5% by weight, relative to the total weight of the composition.

Preferably, the polymer(s) bearing sugar unit(s)/hydrophobic silica aerogel particles weight ratio varies from 0.1 to 10, better still from 0.2 to 5.

Other Ingredients

The cosmetic composition according to the invention generally comprises a cosmetically acceptable medium, i.e. a medium that is compatible with keratin materials such as the skin of the face or of the body, the lips, the hair, the eyelashes, the eyebrows and the nails.

The compositions according to the invention may be in any of the formulation forms conventionally used, and in particular in the form of an aqueous, alcoholic or aqueous-alcoholic, or oily solution or suspension; a solution or a dispersion of the lotion or serum type; an emulsion, in particular of liquid or semi-liquid consistency, of the O/W, W/O or multiple type; a suspension or emulsion of soft consistency of cream (O/W) or (W/O) type; an aqueous or anhydrous gel, or any other cosmetic form.

These compositions may be packaged in pump-action bottles or in aerosol containers, so as to apply the composition in vaporized (lacquer) form or in the form of a mousse. Such packaging forms are indicated, for example, when it is desired to obtain a spray or a mousse, for treating the hair. In these cases, the composition preferably comprises at least one propellant.

The composition according to the invention may be aqueous or anhydrous. It is preferably aqueous and then comprises water at a concentration preferably ranging from 5% to 98% by weight, especially from 20% to 95% by weight and better still from 50% to 90% by weight, relative to the total weight of the composition.

The composition may also comprise one or more organic solvents that are liquid at 25° C. and 1 atm., and that are in particular water-soluble, such as C1-C7 alcohols; mention may especially be made of C1-C7 aliphatic or aromatic monoalcohols, and C3-C7 polyols and polyol ethers, which may thus be used alone or as a mixture with water. Advantageously, the organic solvent may be chosen from ethanol, isopropanol, benzyl alcohol and mixtures thereof.

The composition according to the invention may also comprise at least one standard cosmetic ingredient, other than the compounds of the invention, and in particular chosen from propellants; plant, mineral, animal or synthetic oils; solid fatty substances and in particular waxes, C8-C40 esters; C8-C40 acids; C8-C40 alcohols; nonionic surfactants, cationic surfactants, anionic surfactants, amphoteric surfactants, zwitterionic surfactants; sunscreens; moisturizers; antidandruff agents; antioxidants; chelating agents; reducing agents; oxidation bases, couplers, oxidizing agents, direct dyes; hair straightening agents such as thiols and alkali metal hydroxides; nacreous agents and opacifiers; plasticizers or coalescers; hydroxy acids; pigments; fillers; silicones and in particular polydimethylsiloxanes (PDMSs); thickeners without sugar unit(s); emulsifiers; conditioning or styling polymers; fragrances; basifying agents or acidifying agents; silanes; crosslinking agents such as polyphenols, aldehydes and DHA. The composition can, of course, comprise several cosmetic ingredients appearing in the above list.

Depending on their nature and the purpose of the composition, the standard cosmetic ingredients can be present in standard amounts which can be easily determined by those skilled in the art and which can be, generally, for each ingredient, between 0.01% and 80% by weight.

The oils may be preferably present in a proportion of from 0.01% to 80% by weight, especially from 0.02% to 40% by weight or even from 0.5% to 20% by weight, relative to the total weight of the composition. They may be carbon-based. Mention may especially be made of hydrogenated or non-hydrogenated plant, animal or mineral oils, saturated or unsaturated, linear or branched, cyclic or aliphatic, hydrocarbon-based synthetic oils, for instance poly-alpha-olefins, in particular polydecenes and polyisobutenes; liquid fatty alcohols such as isostearyl alcohol, octyldodecanol, 2-butyloctanol, 2-hexyldecanol, 2-undecylpentadecanol, oeyl alcohol or linoleyl alcohol; liquid esters such as isopropyl myristate or isopropyl palmitate. Mention may also be made of water-soluble or water-insoluble, organomodified or non-organomodified, volatile or non-volatile silicone oils; fluoro or perfluoro oils; and also mixtures thereof.

The composition may also comprise one or more solid fatty substances, and in particular one or more fatty alcohols, fatty esters and/or fatty acids, other than the above oils, having 8 to 40 carbon atoms; these solid fatty substances may preferably be present in a proportion of from 0.01% to 30% by weight, especially 0.1% to 20% by weight relative to the total weight of the composition. Mention may especially be made of C12-C32, especially C12-C26, linear-chain fatty alcohols, and in particular cetyl alcohol, stearyl alcohol, cetylstearyl alcohol and behenyl alcohol. Mention may also be made of C16-C40 linear-chain or branched-chain fatty acids, and especially 18-methyleicosanoic acid, coconut oil or hydrogenated coconut oil acids; stearic acid, lauric acid, palmitic acid and oleic acid, behenic acid, and mixtures thereof. Preferably, the fatty acids are not salified. Mention may also be made of the linear-chain fatty esters, comprising in total between 8 and 40 carbon atoms, such as myristyl, cetyl or stearyl myristates, palmitates and stearates, alone or as a mixture.

A person skilled in the art will take care to choose the ingredients included in the composition, and also the amounts thereof, such that they do not harm the properties of the compositions of the present invention.

The pH of the composition, if it is aqueous, may be acidic, neutral or alkaline. Preferably, the composition has a pH of between 2 and 11, in particular from 3 to 9, or even 3 to 7.

The cosmetic composition according to the invention may be in the form of a product for caring for, cleansing and/or making up bodily or facial skin, the lips, the eyebrows, the eyelashes, the nails and the hair, an antisun or self-tanning product, a body hygiene product, or a haircare product, especially for caring for, cleansing, styling, shaping or coloring the hair.

The composition especially finds a particularly advantageous use in the field of haircare, especially for holding the hairstyle or shaping the hair, or for the care, cosmetic treatment or cleansing of the hair. The haircare compositions are preferably shampoos, hair conditioners, styling or care gels, care lotions or creams, conditioners, masks, serums, hairsetting lotions, blow-drying lotions, hair styling and fixing compositions such as lacquers or sprays; hair restructuring lotions; lotions or gels for preventing hair loss, antiparasitic shampoos, antidandruff lotions or shampoos, and anti-seborrhea treatment shampoos. The lotions may be packaged in various forms, especially in vaporizers, in pump-action bottles or in aerosol containers so as to apply the composition in vaporized form or in the form of a mousse.

The composition according to the invention finds a particularly advantageous use for caring for, treating and/or cleansing the hair and/or scalp, for example as a shampoo, as conditioner, as a shampoo and/or conditioner post-treatment, or else between two shampooing operations.

It is thus possible, for example, to apply the composition according to the invention to the scalp and/or the hair, directly after shampooing, on wet hair or on dry hair, with or without a leave-on time, with or without heat, said application optionally being followed by rinsing and drying, either at room temperature or with a hairdryer or else with a hair straightener (250° C.) for example.

It is also possible to apply the composition according to the invention to the scalp and/or the hair, directly after conditioning, on wet hair or on dry hair, with or without a leave-on time, with or without heat, said application optionally being followed by rinsing and drying, either at room temperature or with a hairdryer or else with a hair straightener (250° C.) for example.

It is also possible to apply the composition according to the invention to the scalp and/or the hair between two shampooing operations, on wet hair or dry hair.

The cosmetic composition may or may not be rinsed out after having been applied to the keratin materials (hair and/or scalp).

Another subject of the invention is a cosmetic treatment method, especially for making up, caring for, cleansing, coloring or shaping keratin materials, especially bodily or facial skin, the lips, the nails, the hair and/or the eyelashes, comprising the application to said materials of a cosmetic composition according to the invention.

It is in particular a hair treatment method for the care, cosmetic treatment and/or cleansing of the hair and/or of the scalp, and in particular that makes it possible to delay or even prevent regreasing of the hair and/or of the scalp, and thus to space out the shampooing operations.

The invention is illustrated in greater detail in the examples that follow.

### EXAMPLES

The following haircare compositions are prepared (% by weight):

| | |
|---|---|
| | Composition A |
| SILICA SILYLATE (INCI name) | 0.5% |
| Hydroxypropyl methyl cellulose (HPMC) | 0.3% |
| Methocel F 4 M from Dow Chemical | |
| Water | qs 100% |
| | Composition B |
| SILICA SILYLATE (INCI name) | 0.5% |
| Hydroxyethyl cellulose | 0.3% |
| Natrosol 250 HHR PC from Ashland | |
| Water | qs 100% |
| | Composition C |
| SILICA SILYLATE (INCI name) | 0.5% |
| Xanthan gum | 0.3% |
| Rhodicare XC from Rhodia | |
| Water | qs 100% |

The SILICA SILYLATE used is the product sold under the name DOW CORNING VM-2270 AEROGEL FINE PARTICLES by Dow Corning.

The compositions are easy to apply to the hair and the scalp; they make it possible to reduce the regreasing of the hair.

1. A cosmetic composition comprising:
hydrophobic silica aerogel particles and
at least one polymer bearing sugar unit(s).

2. The composition as claimed in claim 1, wherein the hydrophobic silica aerogel particles have a specific surface area per unit of mass (S_{M}) ranging from 500 to 1500 m²/g, preferably from 600 to 1200 m²/g and better still from 600 to 800 m²/g.

3. The composition as claimed in either one of the preceding claims, wherein the hydrophobic silica aerogel particles have an oil absorption capacity, measured at the wet point, ranging from 5 to 18 ml/g, preferably from 6 to 15 ml/g and better still from 8 to 12 ml/g of particles.

4. The composition as claimed in any one of the preceding claims, wherein the hydrophobic silica aerogel particles have a size, expressed as the mean diameter (D[0.5]), of less than 1500 μm, and preferably ranging from 1 to 30 μm, preferably from 5 to 25 μm, better still from 5 to 20 μm and even better still from 5 to 15 μm.

5. The composition as claimed in any one of the preceding claims, wherein the hydrophobic silica aerogel particles have a tapped density p ranging from 0.04 g/cm³ to 0.10 g/cm³ and preferably from 0.05 g/cm³ to 0.08 g/cm³.

6. The composition as claimed in any one of the preceding claims, wherein the hydrophobic silica aerogel particles have a specific surface area per unit of volume (S_{V}) ranging from 5 to 60 m²/cm³, preferably from 10 to 50 m²/cm³ and better still from 15 to 40 m²/cm³.

7. The composition as claimed in any one of the preceding claims, wherein the hydrophobic silica aerogel particles are silylated silica aerogel particles and in particular aerogel particles of hydrophobic silica surface-modified with trimethylsilyl groups.

8. The composition as claimed in any one of the preceding claims, wherein the hydrophobic silica aerogel particles are present in an amount of between 0.01% and 20% by weight, preferably between 0.05% and 10% by weight and preferentially between 0.1% and 5% by weight, relative to the total weight of the composition.

9. The composition as claimed in any one of the preceding claims, wherein the sugar units that may be included in the composition of the polymers are derived from the following sugars: glucose, galactose, arabinose, rhamnose, mannose, xylose, fucose, fructose, anhydrogalactose, galacturonic acid, glucuronic acid, mannuronic acid, galactose sulfate, anhydrogalactose sulfate.

10. The composition as claimed in any one of the preceding claims, wherein the polymers bearing sugar unit(s) are nonionic, anionic, amphoteric or cationic.

11. The composition as claimed in any one of the preceding claims, wherein the polymers bearing sugar unit(s) are chosen from modified or unmodified guar gums, locust bean gums, xanthan gums, starches and celluloses.

12. The composition as claimed in any one of the preceding claims, wherein the polymers bearing sugar unit(s) are present in an amount of between 0.01% and 10% by weight, especially from 0.05% to 5% by weight, preferentially from 0.1% to 1% by weight, or even from 0.1% to 0.5% by weight, relative to the total weight of the composition.

13. The composition as claimed in any one of the preceding claims, wherein the polymer(s) bearing sugar unit(s)/hydrophobic silica aerogel particles weight ratio varies from 0.1 to 10, better still from 0.2 to 5.

14. The composition as claimed in any one of the preceding claims, comprising water at a concentration ranging from 5% to 98% by weight, especially from 20% to 95% by weight and better still from 50% to 90% by weight, relative to the total weight of the composition.

15. The composition as claimed in any one of the preceding claims, being in the form of a haircare product, especially for the care, cosmetic treatment or cleansing of the hair and/or of the scalp.

16. A cosmetic treatment method, especially for making up, caring for, cleansing, coloring or shaping keratin materials, especially bodily or facial skin, the lips, the nails, the hair and/or the eyelashes, comprising the application to said materials of a cosmetic composition as claimed in one of claims 1 to 15.

17. The hair treatment method as claimed in claim 16, for the care, cosmetic treatment and/or cleansing of the hair and/or of the scalp, in particular that makes it possible to delay or even prevent regreasing of the hair and/or of the scalp.
